(19) United States
(12) Patent Application Publication
(10) Pub. No.: US 2021/0037949 A1
(43) Pub. Date: Feb. 11, 2021

(54) APPARATUS AND METHODS FOR CONCEALING VITILIGO

(71) Applicant: Zanderm LLC, West Palm Beach, FL (US)

(72) Inventors: James R. Adelson, Far Rockaway, NY (US); Sara R. Frankel, Suffern, NY (US)

(21) Appl. No.: 16/536,537

(22) Filed: Aug. 9, 2019

(51) Int. Cl. A45D 34/04; A61K 8/35; A61Q 1/02; B43K 5/18; B43K 1/12

(52) U.S. Cl. CPC A45D 34/042; A61K 8/35; A61Q 1/02; A45D 2200/1045; B43K 1/12; A61K 2800/87; A61K 2800/43; B43K 5/18

(57) ABSTRACT

Apparatus for concealing vitiligo is provided. The apparatus may include a cosmetic marker for applying a solution for concealing vitiligo. The cosmetic marker may support enhanced flow of the solution from the marker onto a portion of depigmented skin. The cosmetic marker may include a marker barrel. The cosmetic marker may also include a fibrous reservoir positioned in the marker barrel. The fibrous reservoir may be saturated with the solution. The fibrous reservoir may have a density of preferably between 0.12 g/cc-0.14 g/cc. The cosmetic marker may also include a polyester nib in fluid communication with the fibrous reservoir. The polyester nib may have a porosity of between 65%-70%. The cosmetic marker may also include the solution. The solution may include a colorant and dihydroxyacetone.

### FIELD OF TECHNOLOGY

This invention relates to apparatus and methods for concealing vitiligo. More specifically, this invention relates to concealing vitiligo using an applicator that applies a camouflaging solution onto a portion of skin affected by vitiligo.

This invention additionally relates to apparatus and methods for concealing unwanted tan lines. More specifically, this invention relates to concealing unwanted tan lines using an applicator that applies a camouflaging solution onto a portion of skin.

### BACKGROUND OF THE DISCLOSURE

Vitiligo is a condition that causes skin depigmentation on portions of a human body. Vitiligo depigmentation typically occurs on the extremities of the body, such as the face, mouth, eyes, nostrils, hands and feet. Typically, the depigmentation is manifest in patches that cover small or large areas of the body.

Skin depigmentation from vitiligo is caused by the death of skin cells responsible for skin pigmentation. The skin cell death creates a completely white area on an area of skin which was previously pigmented.

Because vitiligo completely depigments the skin, there is a stark contrast between the white, depigmented skin, and the surrounding pigmented skin. As a result, these symptoms are visible to onlookers when the affected area is exposed.

Current methods used to treat vitiligo include phototherapy, immune mediators, complete skin-depigmentation and/or melanocyte transplantation. These methods are intrusive because they include medications, steroids, surgery, and/or exposure to phototherapy.

Other methods available to conceal vitiligo do not treat the underlying cause of the disease, such as methods using creams or makeup to camouflage the depigmented skin. The camouflaging effect of these creams and makeup typically lasts anywhere from 6-12 hours. Thus, the camouflaging effect is temporary and can be easily removed.

It would be desirable, therefore, to provide a method for camouflaging vitiligo symptoms that both conceals the depigmented skin and lasts substantially longer than 6-12 hours.

The application of a solution to depigmented skin should be precise. This is because the application of more or less solution could result in overly darkened skin or pigmenting skin that is naturally pigmented. Therefore, an applicator for applying a solution to a depigmented area should be easy to control, and dispense a predictable amount of solution.

It would be desirable, therefore, to provide apparatus and methods for camouflaging vitiligo that include an applicator that precisely and uniformly applies a solution to depigmented skin.

Furthermore, it is desirable to provide apparatus for camouflaging unwanted tan lines.

For many people, a sun tan creates a fashionable and desirable appearance. A sun tan can be acquired through prolonged skin exposure to the sun, or through application of one or more sunless tanning solutions.

However, a sun tan is acquired only on portions of the body that were exposed to the sun or tanning solution. Portions of the body that were not exposed to the sun or tanning solution are typically lighter in color relative to exposed portions of the body. This results in the appearance of one or more streaks, spots, lines or other forms of discoloration on the body, also known as ‘tan lines.’

Tan lines are undesirable at least because they create an irregular appearance on the body. Tan lines are also undesirable because they reveal the shape of clothing that was worn during exposure to the sun, such as a bathing suit, shoes, goggles or golfing gloves.

It would be desirable, therefore, to provide a cosmetic applicator that is configured to precisely apply a solution on the body that conceals unwanted tan lines.

Solution applicators that include tan line correction solution are currently available in the market. The Tanee™ Company, located in Bronx, N.Y., has manufactured a tan line corrector in a tube-like body which a user squeezes to dispense tan line correction solution. The Norvell™ Company, located in Alexandria, Tenn., has manufactured a tan line corrector with a bottom that, when twisted, dispenses tan line correction solution.

However, these solution applicators have failed to provide customers with a precise and reliable delivery of tan line correction solution. The Tanee™ and Norvell™ solution applicators include low-density tip applicator that leaks, is messy, and is difficult to control. Additionally, both the Tanee™ and Norvell™ applicators store the tan line correction solution in an open reservoir included in their applicator. The open reservoir has an isotropic fluid flow—i.e., a fluid flow with no directional preference—and has a tendency to oversaturate the sponge tip. Such oversaturation causes unwanted flow of solution from the applicator.

The tan line correction applicator should be easy to control, and dispense a predictable amount of solution. This is because the application of more or less tan line correction solution than necessary will, at best, result in uneven coloring of the body and, at worst, stain clothing. This renders the applicator unsuitable for its intended purpose of creating a uniform coloring on the body.

It would be desirable, therefore, to provide a cosmetic applicator that is able to precisely and uniformly apply a solution to camouflage unwanted tan lines.

### SUMMARY OF THE DISCLOSURE

Apparatus and methods for concealing one or more of vitiligo and unwanted tan lines are provided. The apparatus may include a cosmetic marker for applying a solution for concealing vitiligo. The cosmetic marker may support enhanced flow of the solution from the marker onto a portion of depigmented skin. The cosmetic marker may include a marker barrel. The cosmetic marker may include a reservoir positioned in the marker barrel. The reservoir may be a fibrous reservoir or any other reservoir described herein. The fibrous reservoir may be saturated with the solution.

The fibrous reservoir may have a density of between 0.12 g/cc-0.13 g/cc, 0.12 g/cc-0.14 g/cc, 0.12 g/cc-0.15 g/cc, 0.12 g/cc-0.16 g/cc, 0.12 g/cc-0.17 g/cc or 0.12 g/cc-0.18 g/cc. The fibrous reservoir may have a density of approximately between 0.12 g/cc-0.13 g/cc, 0.12 g/cc-0.14 g/cc, 0.12 g/cc-0.15 g/cc, 0.12 g/cc-0.16 g/cc, 0.12 g/cc-0.17 g/cc, or 0.12 g/cc-0.18 g/cc. For the purposes of the application, the term “approximately” is used to describe +/−2% of a value.

The cosmetic marker may include a nib. The nib may be a polyester nib or any other nib described herein. The polyester nib may be in fluid communication with the fibrous reservoir. The polyester nib may have a porosity of between 65%-70%, 65%-75% or 60%-80%. The polyester nib may have a porosity of between approximately 65%-70%, 65%-75% or 60%-80%.

The cosmetic marker may include the solution. The solution may include a colorant and dihydroxyacetone (“DHA”). The solution may include any other ingredients or combinations of ingredients described herein. The solution may comprise a specific gravity of between 0.90-1.2, a pH of between 2.5 and 3.7, and a viscosity using a (Spindle #3/100 RPM/1 min) of between 4-23 centipoise. The solution may comprise different specific gravity, pH and viscosity values as set forth at Table 1 below.

The colorant may be a bronzer or any other colorant described below.

The polyester nib may be formed from pressed fibers. The polyester nib may include one or both of resin, porous polyethylene, or any other suitable material(s).

The fibrous reservoir may deliver the solution to the nib through capillary action. The solution may be held in suspension within the fibrous reservoir. The fibrous reservoir may include a shell and a fibrous filling. The fibrous filling may be selected from a group consisting of felt and polyester. A flow of solution from the fibrous reservoir to the nib may be anisotropic. The fibrous reservoir may define channels extending towards the nib. The channels may not be omnidirectional.

The nib may substantially retain its shape when depressed on a surface. Movement of the nib along the portion of the depigmented skin may result in an application of a regulated amount of the solution to the skin.

In some embodiments, the apparatus described above may be used to conceal unwanted tan lines.

### BRIEF DESCRIPTION OF THE DRAWINGS

The objects and advantages of the invention will be apparent upon consideration of the following detailed description, taken in conjunction with the accompanying drawings, in which like reference characters refer to like parts throughout, and in which:

FIG. 1 shows illustrative apparatus for use with the systems and methods of the invention;

FIGS. 2A-2B shows illustrative apparatus for use with the systems and methods of the invention;

FIG. 3 shows illustrative apparatus for use with the systems and methods of the invention;

FIG. 4 shows illustrative apparatus for use with the systems and methods of the invention;

FIG. 5 shows illustrative apparatus for use with the systems and methods of the invention;

FIG. 6 shows illustrative apparatus for use with the systems and methods of the invention;

FIG. 7 shows illustrative apparatus for use with the systems and methods of the invention;

FIG. 8 shows illustrative apparatus for use with the systems and methods of the invention;

FIG. 9 shows illustrative apparatus for use with the systems and methods of the invention;

FIG. 10 shows illustrative apparatus for use with the systems and methods of the invention;

FIG. 11 shows illustrative apparatus for use with the systems and methods of the invention;

FIG. 12 shows illustrative apparatus for use with the systems and methods of the invention;

FIG. 13 shows illustrative apparatus for use with the systems and methods of the invention; and

FIG. 14 shows illustrative apparatus for use with the systems and methods of the invention.

### DETAILED DESCRIPTION OF THE DISCLOSURE

Apparatus for an applicator configured to accurately apply a solution for concealing vitiligo and/or unwanted tan line(s) is provided. In some embodiments, the applicator may be a marker-like applicator, a spring-tension applicator, or any other suitable applicator.

Solution for Concealing Vitiligo and/or Unwanted Tan Lines

The solution for concealing vitiligo and/or unwanted tan lines (hereinafter, ‘the solution’) may include a colorant such as a bronzer, a sunless tanner and/or any other suitable ingredient(s).

The solution may include a colorant, a sunless tanner, a preservative, an emulsifier and/or any other suitable ingredients.

In some embodiments, the solution may include water. In some of these embodiments, the solution may be water-based.

In some embodiments, the solution may include witch hazel. The witch hazel may include phenols such as gallic acid, tannis of the proanthocyanin type, catechins, flavonols, chemicals found in essential oil (carvacrol, eugenol and hexenol), saponins and choline. The witch hazel may be distilled witch hazel. In some of these embodiments, the base of the solution may be witch hazel.

Any suitable colorant may be may be included in the solution. For the purposes of the application, a colorant may be anything that creates color on the skin, such as a bronzer, dye, coloring agent, staining agent, cosmetic coloring and/or pigment.

The colorant may give the skin a darker look, a tanned look, or any other different appearance. The colorant may be formed from natural ingredients. The colorant may include one or more synthetic ingredients.

The colorant included in the solution may affect the immediate coloring of the depigmented, or not tanned, skin area. Thus, the colorant may be used to initially camouflage the depigmented, or not tanned, skin.

One or more colorants may be included in the solution. Exemplary colorants include Black Walnut Extract, caramel color, and a bronzer. The bronzer may be an all-natural bronzer. The bronzer may be an instant bronzer. The bronzer included in the solution may affect the immediate coloring of the depigmented skin area. Thus, the bronzer may be used to initially camouflage the depigmented skin.

Additional exemplary colorants may include one or more synthetic dyes assigned Food, Drug and Cosmetic (hereinafter, “FD&C”) numbers by the United States Food and Drug Administration (hereinafter, “FDA”). Exemplary FD&C colors that may be included in the solution include one or more of Yellow 5 (Color Index (hereinafter, “CI”) 19140), Yellow 6 (CI 15985), Red 33 (CI 17200), Red 40 (CI 16035), Blue 1 (CI 42090) and/or any other suitable FD&C color(s). The synthetic dyes in the cosmetic solution may be selected from the group consisting of Yellow 5 (Color Index (hereinafter, “CI”) 19140), Yellow 6 (CI 15985), Red 33 (CI 17200), Red 40 (CI 16035) and Blue 1 (CI 42090).

Other exemplary colorants include henna, body paint, tattoo marker solution, surgical marker solution, permanent marker solution, permanent or semi-permanent magic marker solution, a cosmetic solution including but not limited to a solution included in one or more of eyeliner, eye shadow, lipstick, lip liner, base, foundation, or blush, that lasts for 1-3 days, 3-5 days, 7-10 days, 10 or more days, or any other suitable number of days or range of days, or any other suitable solution may be used.

Application of a colorant to the skin may result in the painting, dying, and/or staining of the skin.

Any suitable sunless tanner may be included in the solution. For the purposes of the application, a sunless tanner may be a product that creates a tanned appearance on the skin without exposing the skin to either the sun or another source of ultraviolet radiation. An exemplary sunless tanner that may be used is dihydroxyacetone (“DHA”). The DHA may be derived from sugar beets, sugar cane, or through any other suitable method.

In the embodiments in which the solution includes DHA and bronzer, the bronzer may substantially immediately affect a coloring of depigmented, or not tanned, skin when applied to the skin. The DHA may also affect a coloring of depigmented, or not tanned, skin. The coloring affected by the DHA may last longer relative to the coloring affected by the bronzer. Application of the DHA may not automatically affect a change in the skin color. However, application of the DHA to the skin may trigger a chemical reaction with amino acids present in the epidermal layer located at the skin's surface. After the passage of time, such as 2-4 hours, the chemical reaction may begin to create a brown color on the upper layer of the skin. The brown color may continue to darken for the next 24-72 hours. The brown color may typically last anywhere from 3 to 10 or more days, depending on how much DHA was applied and the natural exfoliation rate of the skin.

In the embodiments in which the solution includes DHA and one or more colorants, the colorant may affect the immediate pigmenting of the skin. The DHA may cause a coloring of the skin after the passage of time due a chemical reaction, as detailed above. In some embodiments, when the colorant wears off, the coloring of the skin induced by the DHA may remain, allowing for a longer lasting skin coloring. In some embodiments, as the effect of the colorant decreases on the skin, the DHA coloring of the skin may increase.

The solution may have high or low viscosity. The solution may take any suitable physical form. For example, the solution may take the physical form of an emulsion, cream, solid, stick, waxy solid, ointment, balm, paste, film and/or a watery liquid.

An exemplary appearance of the solution may be a dark brown liquid, a light brown liquid, any other suitable shade of brown or any other suitable color. The solution may have a fragrance to mask its original odor, such as a Tropical Gold Fragrance or any other suitable fragrance.

For example, in some embodiments, the solution may include one or more of water, deionized water, glycerin, DHA, isopentyldiol, Black Walnut Extract, caramel color, phenoxyethanol, citric acid, fragrance, artificial color(s) such as a brown color, one or more preservatives, natural and/or not natural coloring agent(s), and/or any other suitable ingredient(s). In some embodiments, the solution may also include one or more of lycopene, the amino acid Tyrosine, the synthetic melatonin-stimulating hormone analog afamelanotide and/or bronzer(s).

In other embodiments, the solution may include one or more of water, glycerin, DHA, propylene glycol, phenoxyethanol, propandiol, disodium edta, a bronzer and/or any other suitable ingredient(s).

In yet other embodiments, the solution may include one or more of DHA and a colorant. The colorant may be a bronzer or any other colorant described herein. The solution may also include water, glycerin, propylene glycol, phenoxyethanol, propandiol, disodium edta and/or any other suitable ingredient(s) or color(s).

In yet other embodiments, the solution may include witch hazel, DHA, and one synthetic dye assigned an FD&C color. In yet other embodiments, the solution may include witch hazel, DHA, and two synthetic dyes assigned FD&C colors. In yet other embodiments, the solution may include witch hazel, DHA, and three or more synthetic dyes assigned FD&C colors. Exemplary FD&C color(s) that may be included in the solution are yellow 5, red 40, blue 1, red 33 and yellow 6.

In some embodiments, the DHA may comprise 7% to 22% of the solution. The FD&C color(s) may comprise 2% of the solution, and most, or all, of the remaining solution may comprise witch hazel. For example, the witch hazel may comprise 60%, 70%, 80%, or 90% of the solution, or any other integer in between. In some embodiments, the solution may also include a fragrance.

The solution in the cosmetic marker may comprise between 7% and 22% DHA. The solution in the cosmetic marker may comprise between 60% and 90% witch hazel.

The amount of DHA included in the solution may affect darkening properties of the solution. For example, a solution that creates a dark color on the skin after the passage of time, as detailed above, may include more DHA in comparison to a solution that creates a lighter color on the skin after the passage of time.

In other embodiments, 2%, 10%, 20%, 30% or 40% of the solution may be comprised of witch hazel. In some of these embodiments, the solution may also include DHA, FD&C color(s), a preservative, water, and in some embodiments, a fragrance.

In exemplary embodiments, the solution may include 81% witch hazel, 17% DHA, and 2% synthetic dye. Exemplary synthetic dye may include FD&C color(s), such as one or more of yellow 5, red 40, blue 1, red 33, yellow 6.

All numerical values set forth in the specification and claims shall be understood to be modified by the term ‘about.’ Additionally, unless the meaning is clearly to the contrary, all ranges set forth in the specification and the claims are deemed to be inclusive of their end points.

Application of the solution to an area affected by vitiligo, or an area of skin that is not tanned, may result in a two step process. The first step may be the immediate camouflage of the skin by the colorant. The second step may be the browning of the skin resultant from the chemical reaction triggered by the DHA.

Thus, the application of the solution may camouflage, blend, cover, disguise, and/or conceal the depigmented, or not tanned, area of skin, resulting in a more homogenous appearance of the skin. The camouflaging, blending, covering, disguising, and/or concealing of the skin area may last for three to ten, or more, days.

The solution may additionally or alternatively be used to create an appearance of freckles on areas of depigmented skin. For example, a person with freckled skin, who has been affected by vitiligo, may desire to recreate the appearance of freckles on portions of depigmented skin. The solution may be applied to many small areas of the depigmented skin to create a freckled look. This application may assist in concealing the depigmentation of the skin.

In some embodiments, a person may apply a first solution in accordance with the invention to an area of depigmented skin. The first solution may include a first amount of DHA. The person may then apply a second solution in accordance with the invention to small areas of the skin that has already been treated by the first solution. The second solution may have a second amount of DHA. The application of the first solution may conceal the depigmentation of the skin. The application of the second solution may create a freckled look on the skin. The first amount may be substantially equal to the second amount. The first amount may be less than the second amount. The first amount may be greater than the second amount.

### Applicators

The solution described above may be contained in any suitable applicator. Any applicator known to those skilled in the art may be used to hold the solution. For example, the solution may be contained in a spring-tension applicator, a spray bottle including a pump, a roller-ball bottle, a dispensing tube, a vial with a cap including an applicator, a squeeze bottle, a lipstick-like holder, a jar, a marker, a pen, a cream dispenser, or any other suitable container or cosmetic applicator.

In the embodiments in which the solution is held in a reservoir, the reservoir may be a liquid reservoir, an open reservoir, a fibrous reservoir, or any other suitable reservoir.

### Spring-Tension Applicator

In some embodiments, the applicator may be a spring-tension applicator (hereinafter, ‘spring applicator’). The spring applicator may be any spring applicator known to those skilled in the art, such as the spring applicator produced by Dab-O-Matic Corp., located in Mount Vermon, N.Y.

In some embodiments, the spring applicator may include an applicator head and an applicator barrel. The applicator head may be inserted into, and supported by, the applicator barrel.

The applicator barrel may be any suitable shape and size. For example, the applicator body may comprise one or more cylindrical, rectangular, oval, substantially circular, and/or any other suitable shape(s). The applicator barrel may be closed at one end and open at the other end. An inner area of the applicator barrel may be hollow. At least a portion of the hollow area of the applicator barrel may be filled with the solution disclosed herein.

In some embodiments, the applicator barrel may hold the solution in an open reservoir. In some embodiments, the applicator barrel may hold the solution in a fibrous reservoir.

The applicator head may be configured to be inserted into, or onto, the open end of the applicator barrel. Insertion of the applicator head into, or onto, the applicator barrel may form a fluid-tight connection. In some embodiments, the applicator head may be screwed onto the barrel, forming a fluid-tight connection. The fluid-tight connection may ensure that the solution does not leak from the point of connection between the applicator head and the applicator barrel.

In exemplary embodiments, the applicator barrel may be substantially cylindrical. The substantially cylindrical shape of the applicator barrel may include a single diameter or two or more diameters. In some of these embodiments, at least a portion of a first cylindrical shape may be configured to hold the applicator head and at least a portion of a second cylindrical shape may be configured to hold the solution.

In some embodiments, the first cylindrical shape may form a unitary body with the second cylindrical shape. In some embodiments, the first cylindrical shape may be fixedly attached to the second cylindrical shape.

The applicator head may include a cylindrical body and a circular head. The circular head may be located on top of the cylindrical body. In some embodiments, the cylindrical body and the circular head may form a unitary structure. In some embodiments, the cylindrical body may be fixedly attached to the unitary head.

The circular head may have a diameter greater than the diameter of the cylindrical body. In some embodiments, the diameter of the circular head may be equal to a diameter located at the open end of the applicator barrel. In some of these embodiments, insertion of the applicator head into the applicator barrel may position the cylindrical body within the applicator barrel and position the circular head flush on top of the applicator barrel. The top of the circular head may be a substantially solid surface with one or more holes located on the top of the circular head.

The applicator head may include a spring and a spring head. The spring may be located in a hollow portion of the cylindrical body. The spring head may be located on the top of the spring. When the spring is at rest, the spring may urge at least a portion of the spring head into the one or more holes located at the top of the circular head.

When the spring head is urged into the one or more holes, the spring head may form a fluid tight, or substantially fluid tight, seal. Thus, any solution located in the applicator head may be obstructed from leaving the applicator while the spring head is urged into the hole(s).

The spring may be formed from any desirable material, such as one or more of high density polyethylene (“HDPE”), low density polyethylene (LDPE), celcon and/or polypropylene. The spring may be positioned in a hollow inner portion of the cylindrical body.

The top of the circular head may be covered by a covering. The covering may be formed from any desirable material, such as one or more of polyurethane, neoprene, nylon taffeta, mohair, brushed nylon tricot, APL tricot, nylon fleece, compressed polyurethane and/or nylon taffeta over neoprene.

A user of the spring applicator may desire to cover an area of skin with the solution. The area of skin may be depigmented, or have a coloring different from the coloring of surrounding areas of skin. The user may bring the covering into physical contact with the skin area and press the covering into the skin area.

Depression of the covering may depress the spring head located under the covering. Depression of the spring head may depress the spring, moving the spring head downwards in the direction of the cylindrical body. Movement of the spring head downwards may open the one or more holes in the circular head. The opening of the hole(s) in the circular head may allow the solution contained in the applicator barrel to flow through the cylindrical body, through the holes in the circular head, and saturate the covering. The saturated covering may then deliver solution to the area of skin in physical contact with the covering.

Advantages of the spring applicator in accordance with the principles of the invention include the ability to apply a substantially constant amount of solution to a surface. This obtains precise application of solution. Additionally, the predictability of the solution flow may minimize the problem of leaking and dripping. Furthermore, because the spring head maintains the solution in a sealed environment until the spring is depressed, the solution is less likely to dry out with the passage of time.

Exemplary apparatus that may be used to retain and apply a solution in accordance with the invention may include a cosmetic marker. The cosmetic marker may comprise a spring head. The spring head may be attached to a spring and covered by a covering. The cosmetic marker may also include a reservoir. The reservoir may retain a solution comprising witch hazel, dihydroxyacetone (“DHA”) and FD&C color. The reservoir may be an open reservoir. Depression of the spring head may in turn depress the spring. Depression of the spring head may saturate the covering with the solution.

The spring may be formed from one of high density polyethylene (“HDPE”), low density polyethylene (LDPE), celcon and polypropylene. The covering may be formed from at least one of polyurethane, neoprene, nylon taffeta and brushed nylon tricot.

Exemplary methods that may be used to conceal vitiligo in accordance with the invention may comprise depressing a spring head. The spring head may be covered by a covering. Depression of the spring head may saturate the covering with a solution. The solution may include witch hazel, dihydroxyacetone (“DHA”) and FD&C color. The method may also include guiding the saturated covering over a portion of depigmented skin.

The spring head may be attached to a spring. Depression of the spring head may in turn depress the spring and saturate the covering with the solution.

Additional exemplary methods that may be used to conceal vitiligo in accordance with the invention may comprise guiding an applicator tip along a portion of depigmented skin, the applicator tip being saturated with a solution. The solution may include which hazel, dihydroxyacetone (“DHA”) and FD&C color. The solution may be held in an open reservoir. The applicator tip may be formed from at least one of pressed fibers, resin, plastic, polyester, acrylic and/or porous polyethylene.

### Cosmetic Marker

In some embodiments, the applicator may be a marker-like, or pen-like applicator (hereinafter, ‘cosmetic marker’). The cosmetic marker may be any cosmetic marker known to those skilled in the art, such as the cosmetic marker produced by Dri Mark™ Products, Inc., located in Bethpage, N.Y.

In some embodiments, the cosmetic marker may include a marker barrel, a reservoir located inside the marker barrel and a nib in fluid communication with the reservoir. The cosmetic marker may additionally include the solution. The solution may be held in suspension within the reservoir. The reservoir may be a fibrous reservoir or any other suitable reservoir. When the reservoir is a fibrous reservoir, the solution may be delivered by capillary action, through the fibers, to the nib. Movement of the nib along a surface may result in the application of a preferably regulated amount of the solution to the surface. The rate may be regulated at a predetermined rate dependent at least in part on the characteristics of the nib and the reservoir.

The fibrous reservoir may define channels extending towards the nib. The channels may preferably not be omnidirectional.

The fibrous reservoir may have a density of between 0.12 g/cc-0.13 g/cc, 0.12 g/cc-0.14 g/cc, 0.12 g/cc-0.15 g/cc, 0.12 g/cc-0.16 g/cc, 0.12 g/cc-0.17 g/cc or 0.12 g/cc-0.18 g/cc. The fibrous reservoir may have a density of approximately between 0.12 g/cc-0.13 g/cc, 0.12 g/cc-0.14 g/cc, 0.12 g/cc-0.15 g/cc, 0.12 g/cc-0.16 g/cc, 0.12 g/cc-0.17 g/cc, or 0.12 g/cc-0.18 g/cc. For the purposes of the application, the term “approximately” is used to describe +/−2% of a value.

The cosmetic marker may include a nib. The nib may be a polyester nib or any other nib described herein. The polyester nib may be in fluid communication with the fibrous reservoir. The polyester nib may have a porosity of between 65%-70%, 65%-75% or 60%-80%. The polyester nib may have a porosity of between approximately 65%-70%, 65%-75% or 60%-80%.

Exemplary attributes for the solution in accordance with the invention that may be used in a cosmetic marker are included in Table 1 below. The attributes described below may apply to any solution described herein that may be used to conceal vitiligo and/or tan lines.

TABLE 1

| | | Exemplary |
|---|---|---|
| Attribute | Exemplary Ranges | Value |
| Specific Gravity | 0.95-1.15, 0.90-1.2, 1-1.10 | 1.06 |
| pH | 2.8-3.5, 2.5-3.7, 3-3.3 | 2.85 |
| Viscosity (Spindle # 3/100 | 5-20 cps, 7-17 cps, | 14 cps |
| RPM/1 min) | 4-23 cps, 10-15 cps | |

The cosmetic marker may include a marker barrel, a plug, a reservoir, a nib and a cap. In some embodiments, the reservoir may be a fibrous reservoir.

The marker barrel may be formed from glass, aluminum, plastic, or any other suitable material. The marker barrel may include a front end and a back end. The marker barrel may be hollow in the middle and include an open space in one or more of the front end and the back end. In some embodiments, the plug may be inserted in the open space located at the back end of the marker barrel.

The reservoir may be positioned inside the marker barrel. In some embodiments, the reservoir may be inserted into the marker barrel through the open space located at the back end of the marker barrel. For example, a robotic arm may thrust the reservoir into the plastic barrel. A plug may be subsequently inserted into the open space at the back end of the marker barrel, holding the reservoir in place.

The shape of the reservoir may be the same as, or substantially similar to, an elongated cylinder, an elongated rectangle, or any other suitable geometric shape.

The reservoir may comprise a shell filled with a porous, absorbent and/or fibrous filling. The shell may be described herein as having a tubular shape. However, it should be noted that the shell may have any other suitable shape, such as a rectangular shape or any other suitable geometric shape.

Some embodiments may include a porous, absorbent and/or fibrous reservoir without a shell.

In some embodiments, the tubular shell may be formed from non-rigid material. In other embodiments, the tubular shell may be formed from rigid material.

The tubular shell may be filled with a filling, such as a fibrous filler. The filling may be densely packed in the tubular shell. The filling may be made from felt, polyester and/or any other suitable material. Exemplary polyester included in the tubular shell includes a densely packed highly porous polyester fiber. In some embodiments, the filling may include a set of tubes wrapped in plastic.

The filling included in the reservoir may be saturated with the solution. The filling may retain the solution in suspension.

In some embodiments, a vacuum pump may suck the solution into needles. Each needle may be then be pushed by a robotic arm into a reservoir and inject the solution into the filling, saturating the filling with the solution.

The flow of solution in the reservoir may be anisotropic. In other words, the flow of the solution in the reservoir may have a directional preference. The directional preference may be along the longitudinal axis of the marker barrel.

A nib may be inserted through the open space located at the forward end of the marker barrel. For example, in some embodiments, the nib may be pushed into the open space at the forward end of the marker and held in place by a tension fit. This may also bring the nib into physical contact with the reservoir.

In exemplary embodiments, mechanical vibration and/or pressed air may drive a conveyer belt carrying nibs. The nibs may fall into the clutch of a mechanical claw. The mechanical claw may drop the nibs onto a circular pusher. A circular pusher may force each nib into a marker barrel. The force from the pusher may cause the nib and the marker barrel to interlock, and to bring the nib into physical contact with the reservoir.

The impact between the nib and the reservoir resultant from the insertion of the nib into the marker barrel may flood the nib with solution retained in the reservoir. In some embodiments, the reservoir may deliver the solution to the nib through capillary action. In some embodiments, the force of gravity may also assist in delivering solution from the fibrous reservoir to the nib.

The nib may be porous, oriented and/or include many channels. Exemplary material used to form the nib includes pressed fibers, resin, plastic, polyester, acrylic and/or porous polyethylene. For example, the nib may be formed from highly-porous, oriented, polyester fiber. The physical composition of the nib may assist the nib in absorbing solution from the reservoir by capillary action.

In some embodiments, the nib may be relatively dense and retain its shape, or substantially retain its shape, when depressed on a surface. It should be noted that the nib may be substantially denser than laminated polyester foam. Movement of the nib along a surface results in an application of a regulated amount of solution to the surface.

The viscosity of the solution may be matched up with the porosity of the nib and the density of the fibrous filler. This matching may be used to ensure that the solution works in an optimal capillary reaction with the nib and the fibrous filler.

The nib may be held in a stationary position on the marker barrel. Alternatively, the nib may be retractable.

Movement of the nib along a surface, such as an area of depigmented skin or skin that is not tanned, may result in the nib dispensing solution on the surface. The amount of solution dispensed by the nib may be substantially constant. In some embodiments, a user may not be able to adjust the amount of solution being dispensed from the nib. In other embodiments, a user may be able to adjust the amount of solution being dispensed from the nib. Such adjustment may be accomplished using methods that are known in the art.

The marker barrel may also include a cap. The cap may be removably secured to the forward end of the marker barrel. The cap may protect the nib from being deformed and/or drying out.

Advantages of the cosmetic marker in accordance with the principles of the invention include the ability to apply a substantially constant amount of solution to a surface. This results in the precise application of solution, because of the predictability of the solution flow. Additionally, the nib of the cosmetic marker in accordance with the invention retains the solution in a manner that eliminates the problem of leaking and dripping.

The invention may include methods of applying a solution onto depigmented skin. An exemplary method comprises guiding a nib along a portion of depigmented skin. The nib may be formed from resin and saturated with a solution including witch hazel, dihydroxyacetone (“DHA”) and FD&C color. The nib may be saturated with the solution by capillary action from a fibrous reservoir. The nib may deliver a substantially constant amount of the solution onto the skin.

In exemplary embodiments, the cosmetic marker may include a nib and a fibrous reservoir. The apparatus may also include a solution comprising, dihydroxyacetone (“DHA”) and a synthetic dye. Some embodiments of the solution may also include witch hazel. The fibrous reservoir may be saturated with the solution. The synthetic dye may be a dye assigned a FD&C number as described above.

The invention may include a method for concealing vitiligo. The method may include guiding a nib along a portion of depigmented skin. The nib may be formed from resin and saturated with a solution. The solution may include witch hazel, dihydroxyacetone (“DHA”) and a synthetic dye assigned a Food, Drug and Cosmetic (“FD&C”) number. The nib may be saturated with the solution by capillary action from a fibrous reservoir. A portion of the nib and the fibrous reservoir may be positioned in a marker barrel.

The invention may include a method for concealing vitiligo. The method may include guiding a nib along a portion of depigmented skin. The nib may be a polyester nib, or any other type of nib described herein. The nib may have a porosity of between 60%-80%, or any other suitable range described herein. The nib may be saturated with a solution. The solution may include a bronzer and dihydroxyacetone (“DHA”). The solution may include any other ingredient or combination of ingredients described herein for concealing vitiligo. The solution may have a Specific Gravity of between 0.90-1.2, a pH of between 2.5 and 3.7, and a Viscosity using a (Spindle #3/100 RPM/1 min) of between 4-23 centipoise. The nib may be saturated with the solution by capillary action from a reservoir. The reservoir may be a fibrous reservoir. The fibrous reservoir may have a density of between 0.18 g/cc-0.12 g/cc, or any other suitable range described herein.

The invention may include a method of making a cosmetic marker. The method may comprise sucking up a solution including dihydroxyacetone (“DHA”) and one or more synthetic dyes assigned a Food, Drug and Cosmetic (“FD&C”) number, into a needle. Some embodiments of the solution may also include witch hazel. The method may also include inserting the needle into a fibrous reservoir. The method may also include injecting the solution into the fibrous reservoir. The method may further include inserting the fibrous reservoir into a pen barrel. The method may additionally include pushing a nib onto the pen barrel. Pushing the nib onto the pen barrel may place the nib into physical contact with the fibrous reservoir and flood the nib with the solution through capillary action.

The invention may include a method of making a cosmetic marker. The method may include sucking up a solution including a colorant and dihydroxyacetone (“DHA”) into a needle. The solution may include any other ingredient or combination of ingredients described herein for concealing vitiligo. The solution may have a specific gravity of between 0.90-1.2, a pH of between 2.5 and 3.7, and a viscosity using a (Spindle #3/100 RPM/1 min) of between 4-23 centipoise. The method may also include inserting the needle into a fibrous reservoir having a density of between 0.18 g/cc-0.12 g/cc or any other suitable range described herein. The method may further include injecting the solution into the fibrous reservoir. The method may additionally include positioning a polyester nib having a porosity of between 60%-80%, or any other suitable range described herein, on the cosmetic marker such that the nib is in fluid communication with the fibrous reservoir. The method may further include inserting the fibrous reservoir into a barrel of the cosmetic marker. The positioning of the polyester nib may include pushing the nib into the barrel. The pushing may place the nib into physical contact with the fibrous reservoir and flood the nib with the solution through capillary action.

### FIGURES

Illustrative embodiments of apparatus and methods in accordance with the principles of the invention will now be described with reference to the accompanying drawings, which form a part hereof. It is to be understood that other embodiments may be utilized and structural, functional and procedural modifications may be made without departing from the scope and spirit of the present invention.

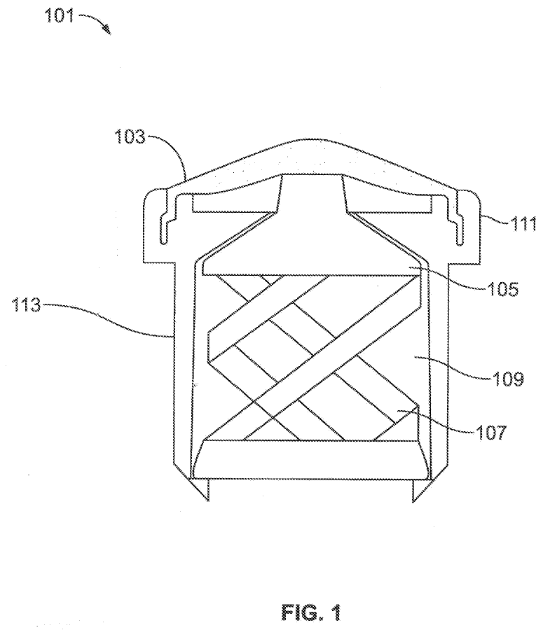

FIG. 1 illustrates exemplary applicator head 101. Exemplary applicator head 101 may include circular head 111 and cylindrical body 113.

Cylindrical body 113 may enclose hollow area 109. Hollow area 109 may be in fluid communication with an applicator barrel (not shown). Thus, the solution held in the applicator barrel may flow freely between the applicator barrel and hollow area 109.

Circular head 111 may be covered by covering 103. Circular head may have an opening with a diameter D.

Spring head 105 may be attached to spring 107. Spring head may include a top portion and a bottom portion. The top portion may cylindrical or substantially cylindrical. The bottom portion may be a frusto-conical shape. Spring 107 may be positioned in cylindrical body 113. When spring 107 is at rest, spring 107 may urge spring head 105 into open area D.

The urging of spring head 105 into open area D may position the top portion of spring head 105 against open area D, creating a fluid-tight, or substantially fluid-tight, seal around open area D. The urging of spring head 105 may also position the bottom portion of spring head 105 flush against a portion of open space 119. The positioning of the bottom portion of spring head 105 flush against a portion of open space 119 may create an additional fluid-tight, or substantially fluid-tight, seal. The creation of the fluid-tight, or substantially fluid-tight, seals may obstruct the flow of any fluid located in hollow area 109 through open area D.

Depression of covering 103 may in turn depress spring head 105. Spring head 105, when depressed, may move downward relative to open area D, compressing spring 107.

Spring head 105 may have diameter G. Hollow area 109 may have diameter B′. Diameter B′ may be larger than diameter G. Thus, the depression of spring head 105 may create fluid communication between hollow area 109 and covering 103. In the event that the solution is retained in hollow area 109, fluid communication between hollow area 109 and covering 103 may result in the saturation of covering 103 with the solution.

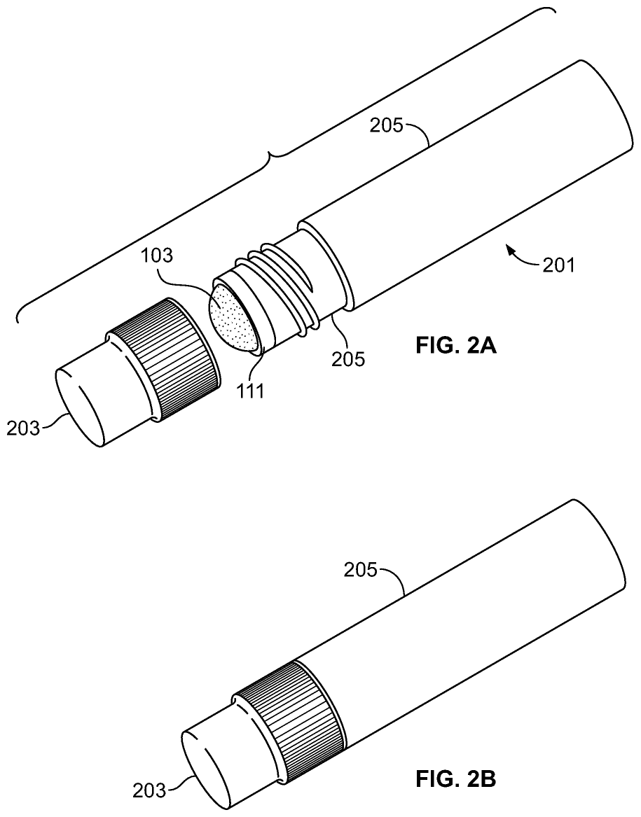

FIG. 2A illustrates spring applicator 201 for use with the principles of the invention. Spring applicator 201 includes applicator barrel 205, applicator head 111 inserted into applicator barrel 205, and covering 103 located on top of applicator head 111. Cap 203 is also illustrated in FIG. 2A. Cap 203 is configured to be removably attached to applicator barrel 205.

FIG. 2B illustrates a cap 203 removably attached to applicator barrel 205.

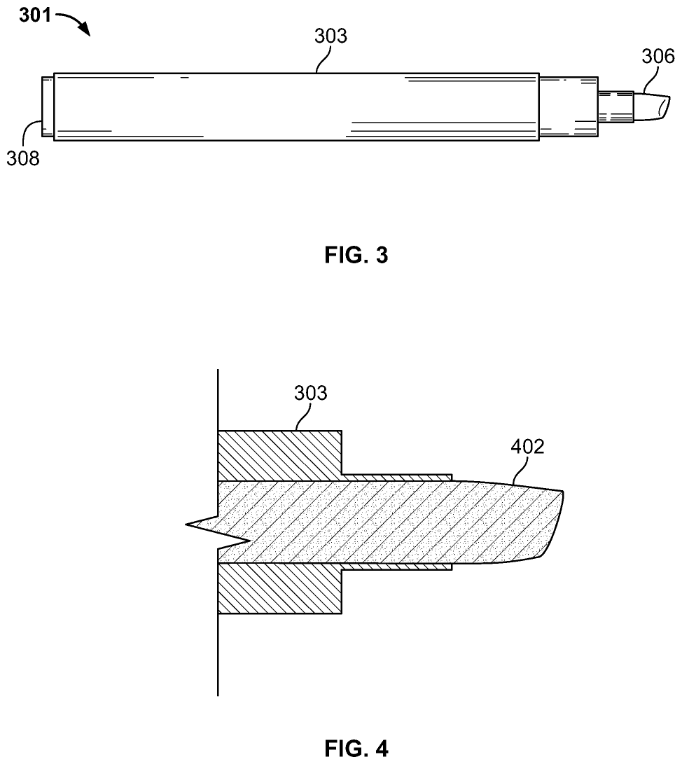

FIG. 3 illustrates cosmetic marker 301 for use with the principles of the invention. The cosmetic marker illustrated in FIG. 3 includes marker barrel 303, nib 306 and plug 308. Nib 306 may be used to dispense the solution onto a portion of depigmented skin, or onto a portion of skin that is not tanned.

FIG. 4 illustrates a portion of a cosmetic marker for use with the principles of the invention. FIG. 4 illustrates nib 402 inserted into marker barrel 303. Nib 402 may be used to dispense the solution onto a portion of depigmented skin, or onto a portion of skin that is not tanned.

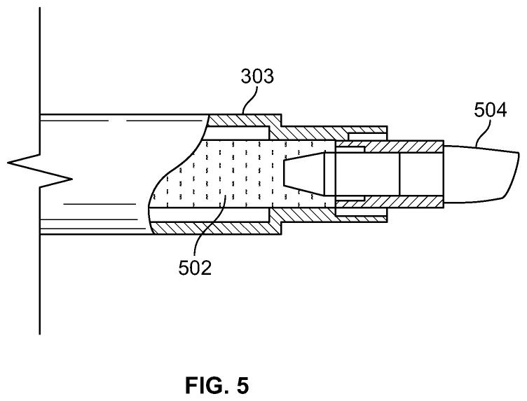

FIG. 5 illustrates another portion of a cosmetic marker for use with the principles of the invention. FIG. 5 illustrates nib 504 inserted into marker barrel 303. Nib 504 is preferably in physical contact, and fluid communication, with reservoir 502. Nib 504 may be used to dispense the solution onto a portion of depigmented skin, or onto a portion of skin that is not tanned.

Reservoir 502 may be saturated with the solution. Reservoir 502 may hold the solution in suspension. Reservoir 502 may deliver the solution to nib 504 by capillary action.

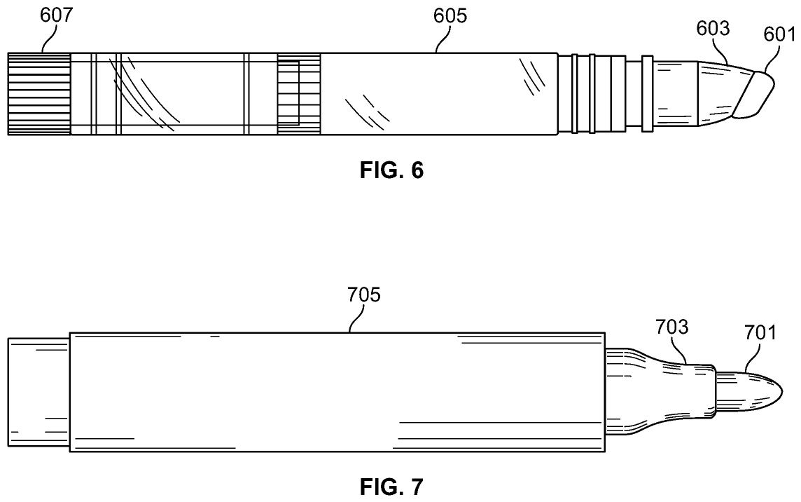

FIG. 6 illustrates another applicator for use with the invention. The applicator illustrated in FIG. 6 may include applicator tip 601 and head 603. Applicator tip 601 may be supported by head 603. The applicator may additionally include body 605. Body 605 may include a reservoir that holds the solution in accordance with the invention. The applicator may further include rotatable end 607. Rotatable end 607, when rotated, may dispense the solution from the reservoir into applicator tip 601. Applicator tip 601 may be used to dispense the solution onto a portion of depigmented skin, or onto a portion of skin that is not tanned.

FIG. 7 illustrates yet another applicator for use with the invention. The applicator illustrated in FIG. 7 may include applicator tip 701 and head 703. Applicator tip 701 may be supported by head 703. The applicator may also include body 705. Body 705 may include a reservoir that holds the solution in accordance with the invention. Applicator tip 701 may be used to dispense the solution onto a portion of depigmented skin, or onto a portion of skin that is not tanned.

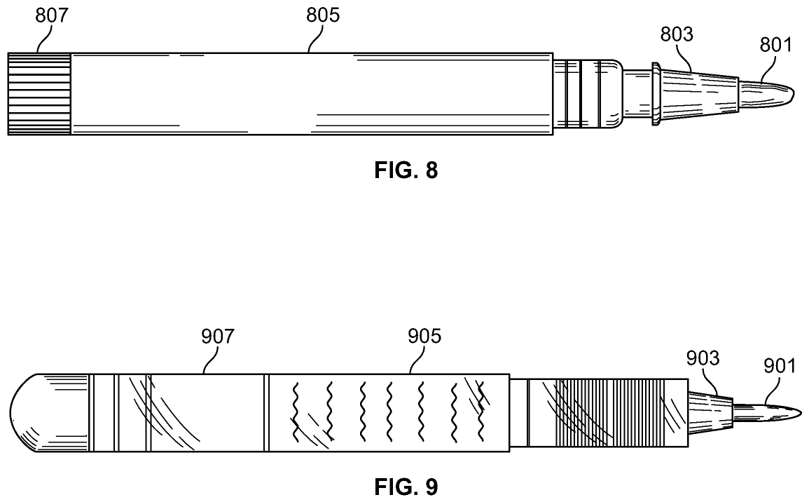

FIG. 8 illustrates yet another applicator for use with the invention. The applicator illustrated in FIG. 8 may include applicator tip 801 and head 803. Applicator tip 801 may be supported by head 803. The applicator may also include body 805. Body 805 may include a reservoir that holds the solution in accordance with the invention. The applicator may additionally include rotatable end 807. Rotatable end 807, when rotated, may dispense the solution from the reservoir into applicator tip 801. Applicator tip 801 may be used to dispense the solution onto a portion of depigmented skin, or onto a portion of skin that is not tanned.

FIG. 9 illustrates yet another applicator for use with the invention. The applicator illustrated in FIG. 9 may include applicator tip 901 and head 903. Applicator tip 901 may be supported by head 903. The applicator may also include body 905. Body 905 may include a reservoir that holds the solution in accordance with the invention. The applicator may additionally include end portion 907. End portion 907 may form a portion of the applicator that does not hold the solution in accordance with the invention. Applicator tip 901 may be used to dispense the solution onto a portion of depigmented skin, or onto a portion of skin that is not tanned.

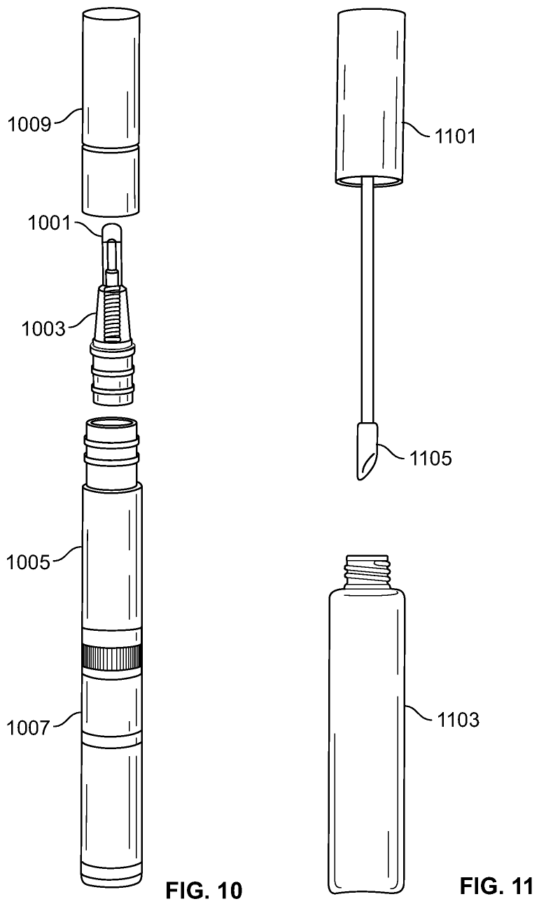

FIG. 10 illustrates yet another applicator for use with the invention. The applicator illustrated in FIG. 10 may include applicator tip 1001 and applicator head 1003. Applicator tip 1001 may be supported by applicator head 1003. Applicator head may fit into body 1005. Body 1005 may include a reservoir that holds the solution in accordance with the invention. The applicator may also include end portion 1007. End portion 1007 may form a portion of the applicator that does not hold the solution. The applicator may additionally include cap 1009.

In some embodiments, end portion 1007 may be rotated around body 1005. Rotation of end portion 1007 around body 1005 may dispense the solution from the reservoir to applicator tip 1001.

Applicator tip 1001 may be used to dispense the solution onto a portion of depigmented skin, or onto a portion of skin that is not tanned.

FIG. 11 illustrates yet another applicator for use with the invention. The applicator illustrated in FIG. 11 may include vial 1003. Vial 1003 may hold the solution in accordance with the invention. The applicator may also include cap 1101. Cap 1101 may screw onto vial 1003. Cap 1101 may include tip 1105. Tip 1105 may be fixedly or removably attached to cap 1101. Tip 1105 may be used to dispense the solution onto a portion of depigmented skin, or onto a portion of skin that is not tanned.

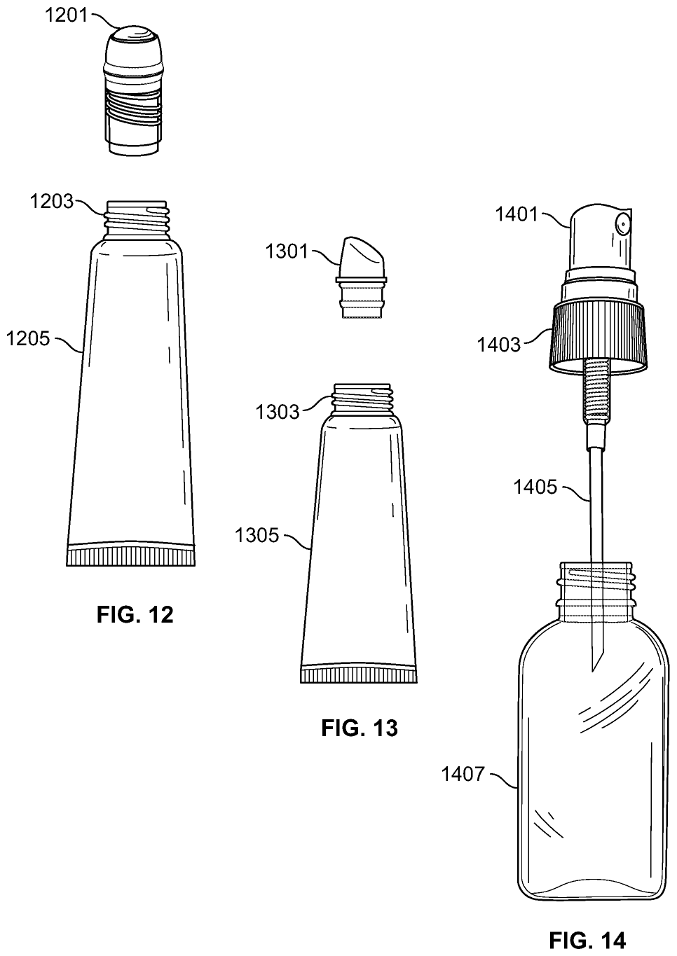

FIG. 12 illustrates yet another applicator for use with the invention. The applicator illustrated in FIG. 12 may include roller ball 1201. Housing including roller ball 1201 may screw onto head 1203. Head 1203 may be located at the top of tube 1205. Tube 1205 may hold the solution in accordance with the invention.

When roller ball 1201 is put into contact with a surface, and moved along the surface, roller ball 1201 may rotate, dispensing onto the surface the solution held in tube 1205. Roller ball 1201 may be used to dispense the solution onto a portion of depigmented skin, or onto a portion of skin that is not tanned.

FIG. 13 illustrates yet another applicator for use with the invention. The applicator illustrated in FIG. 13 may include applicator tip 1301. Applicator tip 1301 may be attached to housing. The housing may screw onto tube head 1303. Tube head 1303 may be located at the top of tube 1305. Tube 1305 may hold the solution in accordance with the invention.

In some embodiments, squeezing tube 1305 may dispense the solution into applicator tip 1301. Applicator tip 1301 may be used to dispense the solution onto a portion of depigmented skin, or onto a portion of skin that is not tanned.

FIG. 14 illustrates yet another applicator for use with the invention. The applicator illustrated in FIG. 14 may include spray head 1401. Spray head 1401 may be attached to housing 1403. Housing 1403 may support both spray head 1401 and tube 1405. Housing 1403 may screw onto bottle 1407. Bottle 1407 may hold the solution in accordance with the invention.

When spray head 1401 is depressed, tube 1405 may draw the solution from bottle 1407 and spray the solution out of spray head 1401. Spray head 1401 may be used to dispense the solution onto a portion of depigmented skin, or onto a portion of skin that is not tanned.

It should be noted that the applicators illustrated in FIGS. 1-14 for use with the invention are for exemplary purposes only. An applicator for use with the invention is not limited in any way to the geometric shape and/or size of the applicators illustrated in FIGS. 1-14. Instead, any suitable shape and/or size of apparatus illustrated in FIGS. 1-14 may be used with the systems and methods of the invention.

Thus, apparatus for markers or applicators and methods for concealing vitiligo and/or unwanted tan lines have been provided. Persons skilled in the art will appreciate that the present invention can be practiced in embodiments other than the described embodiments, which are presented for purposes of illustration rather than of limitation.

1. A cosmetic marker for applying a solution for concealing vitiligo, the cosmetic marker supporting enhanced flow of the solution from the marker onto a portion of depigmented skin, the cosmetic marker comprising:
a marker barrel;
a fibrous reservoir positioned in the marker barrel, the fibrous reservoir being saturated with the solution and having a density of between 0.12 g/cc-0.14 g/cc;
a polyester nib in fluid communication with the fibrous reservoir, the polyester nib having a porosity of between 65%-70%; and
the solution, the solution including a colorant and dihydroxyacetone (“DHA”).

2. The cosmetic marker of claim 1 wherein the colorant is a bronzer.

3. The cosmetic marker of claim 1 wherein the solution comprises a specific gravity of between 0.90-1.2, a pH of between 2.5 and 3.7, and a viscosity using a (Spindle #3/100 RPM/1 min) of between 4-23 centipoise.

4. The cosmetic marker of claim 1 wherein the polyester nib is formed from pressed fibers.

5. The cosmetic marker of claim 1 wherein the polyester nib includes one or both of resin and porous polyethylene.

6. The cosmetic marker of claim 1 wherein the fibrous reservoir delivers the solution to the nib through capillary action.

7. The cosmetic marker of claim 1 wherein the solution is held in suspension within the fibrous reservoir.

8. The cosmetic marker of claim 1 wherein the fibrous reservoir includes a shell and a fibrous filling.

9. The cosmetic marker of claim 8 wherein the fibrous filling is selected from a group consisting of felt and polyester.

10. The cosmetic marker of claim 1 wherein a flow of solution from the fibrous reservoir to the nib is anisotropic.

11. The cosmetic marker of claim 1 wherein the nib substantially retains its shape when depressed on a surface.

12. The cosmetic marker of claim 1 wherein movement of the nib along the portion of the depigmented skin results in an application of a regulated amount of the solution to the skin.

13. The cosmetic marker of claim 1 wherein:
the fibrous reservoir defines channels extending towards the nib; and
the channels are not omnidirectional.

14. A cosmetic marker for applying a solution for concealing vitiligo, the cosmetic marker supporting enhanced flow of the solution from the marker onto a portion of depigmented skin, the cosmetic marker comprising:
a marker barrel;
a fibrous reservoir positioned in the marker barrel, the fibrous reservoir being saturated with the solution and having a density of between 0.12 g/cc-0.18 g/cc;
a polyester nib in fluid communication with the fibrous reservoir, the polyester nib having a porosity of between 60%-80%; and
the solution, the solution including a colorant and dihydroxyacetone (“DHA”), wherein the solution comprises a specific gravity of between 0.90-1.2, a pH of between 2.5 and 3.7, and a viscosity using a (Spindle #3/100 RPM/1 min) of between 4-23 centipoise.

15. The cosmetic marker of claim 14 wherein:
the fibrous reservoir defines channels extending towards the nib; and
the channels are not omnidirectional.

16. The cosmetic marker of claim 14 wherein the fibrous reservoir delivers the solution to the nib through capillary action.

17. A method of making a cosmetic marker, the method comprising:
sucking up a solution including a colorant and dihydroxyacetone (“DHA”) into a needle, the solution comprising a specific gravity of between 0.90-1.2, a pH of between 2.5 and 3.7, and a viscosity using a (Spindle #3/100 RPM/1 min) of between 4-23 centipoise;
inserting the needle into a fibrous reservoir having a density of between 0.12 g/cc-0.18 g/cc;
injecting the solution into the fibrous reservoir; and
positioning a polyester nib having a porosity of between 60%-80% on the cosmetic marker such that the nib is in fluid communication with the fibrous reservoir.

18. The method of claim 17 further comprising inserting the fibrous reservoir into a barrel of the cosmetic marker.

19. The method of claim 18 wherein the positioning of the polyester nib includes pushing the nib into the barrel, the pushing placing the nib into physical contact with the fibrous reservoir and flooding the nib with the solution through capillary action.

20. A method for concealing vitiligo, the method comprising:
guiding a polyester nib along a portion of depigmented skin, the nib having a porosity of between 60%-80% and being saturated with a solution, the solution including a bronzer and dihydroxyacetone (“DHA”), the solution comprising a Specific Gravity of between 0.90-1.2, a pH of between 2.5 and 3.7, and a Viscosity using a (Spindle #3/100 RPM/1 min) of between 4-23 centipoise;
wherein:
the nib is saturated with the solution by capillary action from a fibrous reservoir, the fibrous reservoir having a density of between 0.12 g/cc-0.18 g/cc.
